SEQUENCE LISTING (19 sequences)

SEQ ID NO: 1 - protein, 127 aa, Artificial Sequence
  TH / h.all (universal VH template); (V4L; R48K, _83S; S96T; A54G, N101T; and Y52W)
  misc_feature (1)..(1): Xaa can be any naturally occurring amino acid
  misc_feature (10)..(10): Xaa can be any naturally occurring amino acid
  misc_feature (28)..(30): Xaa can be any naturally occurring amino acid
  misc_feature (32)..(38): Xaa can be any naturally occurring amino acid
  misc_feature (40)..(40): Xaa can be any naturally occurring amino acid
  misc_feature (55)..(55): Xaa can be any naturally occurring amino acid
  misc_feature (57)..(66): Xaa can be any naturally occurring amino acid
  misc_feature (73)..(73): Xaa can be any naturally occurring amino acid
  misc_feature (84)..(84): Xaa can be any naturally occurring amino acid
  misc_feature (105)..(116): Xaa can be any naturally occurring amino acid
XQSLEESGGXRLVTPGTPLTLTCTVSGXXXLXXXXXXXMXWVRQAPGKGLEWIGXIXXXXXXXXXXYASWAKXGRFTISKTSSXTTVDLKMTSPTTEDTATYFCXXXXXXXXXXXXWGPGTLVTVSS

SEQ ID NO: 2 - protein, 127 aa, Artificial Sequence
  TK / k.all (universal Vk template); (A2Q; S20T; S46P, Q52L and P69S)
  misc_feature (27)..(38): Xaa can be any naturally occurring amino acid
  misc_feature (56)..(56): Xaa can be any naturally occurring amino acid
  misc_feature (58)..(64): Xaa can be any naturally occurring amino acid
  misc_feature (73)..(73): Xaa can be any naturally occurring amino acid
  misc_feature (81)..(82): Xaa can be any naturally occurring amino acid
  misc_feature (105)..(116): Xaa can be any naturally occurring amino acid
  misc_feature (127)..(127): Xaa can be any naturally occurring amino acid
AQVLTQTPSPVSAAVGGTVTISCQSSXXXXXXXXXXXXLSWFQQKPGQPPKLLIYXAXXXXXXXSTLASGVPXSRFKGSGXXSGTQFTLTISDVQCDDAATYYCXXXXXXXXXXXXFGGGTEVVVKX

SEQ ID NO: 3 - protein, 115 aa, Artificial Sequence
  hwt (wild-type VH chain)
  misc_feature (73)..(73): Xaa can be any naturally occurring amino acid
QSVEESGGRLVTPGTPLTLTCTVSGFSLTDYDMSWVRQAPGRGLEYIASISIIGNTYYASWAKGRFTISKTSXTTVDLKMTSPTSEDTANYFCAILAVGDSETMWGPGTLVTVSS

SEQ ID NO: 4 - protein, 115 aa, Artificial Sequence
  hV4L (VH chain mutant)
  misc_feature (73)..(73): Xaa can be any naturally occurring amino acid
QSLEESGGRLVTPGTPLTLTCTVSGFSLTDYDMSWVRQAPGRGLEYIASISIIGNTYYASWAKGRFTISKTSXTTVDLKMTSPTSEDTANYFCAILAVGDSETMWGPGTLVTVSS

SEQ ID NO: 5 - protein, 115 aa, Artificial Sequence
  hR48k (VH chain mutant)
  misc_feature (73)..(73): Xaa can be any naturally occurring amino acid
QSVEESGGRLVTPGTPLTLTCTVSGFSLTDYDMSWVRQAPGKGLEYIASISIIGNTYYASWAKGRFTISKTSXTTVDLKMTSPTSEDTANYFCAILAVGDSETMWGPGTLVTVSS

SEQ ID NO: 6 - protein, 115 aa, Artificial Sequence
  hY52W (VH chain mutant)
  misc_feature (73)..(73): Xaa can be any naturally occurring amino acid
QSVEESGGRLVTPGTPLTLTCTVSGFSLTDYDMSWVRQAPGRGLEWIASISIIGNTYYASWAKGRFTISKTSXTTVDLKMTSPTSEDTANYFCAILAVGDSETMWGPGTLVTVSS

SEQ ID NO: 7 - protein, 115 aa, Artificial Sequence
  hA54G (VH chain mutant)
  misc_feature (73)..(73): Xaa can be any naturally occurring amino acid
QSVEESGGRLVTPGTPLTLTCTVSGFSLTDYDMSWVRQAPGRGLEYIGSISIIGNTYYASWAKGRFTISKTSXTTVDLKMTSPTSEDTANYFCAILAVGDSETMWGPGTLVTVSS

SEQ ID NO: 8 - protein, 115 aa, Artificial Sequence
  h_83S (VH chain mutant)
QSVEESGGRLVTPGTPLTLTCTVSGFSLTDYDMSWVRQAPGRGLEYIASISIIGNTYYASWAKGRFTISKTSSTTVDLKMTSPTSEDTANYFCAILAVGDSETMWGPGTLVTVSS

SEQ ID NO: 9 - protein, 115 aa, Artificial Sequence
  hS96T (VH chain mutant)
  misc_feature (73)..(73): Xaa can be any naturally occurring amino acid
QSVEESGGRLVTPGTPLTLTCTVSGFSLTDYDMSWVRQAPGRGLEYIASISIIGNTYYASWAKGRFTISKTSXTTVDLKMTSPTTEDTANYFCAILAVGDSETMWGPGTLVTVSS

SEQ ID NO: 10 - protein, 115 aa, Artificial Sequence
  hN101T (VH chain mutant)
  misc_feature (73)..(73): Xaa can be any naturally occurring amino acid
QSVEESGGRLVTPGTPLTLTCTVSGFSLTDYDMSWVRQAPGRGLEYIASISIIGNTYYASWAKGRFTISKTSXTTVDLKMTSPTSEDTATYFCAILAVGDSETMWGPGTLVTVSS

SEQ ID NO: 11 - protein, 112 aa, Artificial Sequence
  kwt (wild-type Vk chain)
AAVLTQTPSPVSAAVGGTVSISCQSSQSVHHNNYLAWFQQKSGQPPKQLIYAASTLAPGVPSRFKGSGSGTQFTLTISDVQCDDAATYYCAGLYNSGSDIYGFGGGTEVVVK

SEQ ID NO: 12 - protein, 112 aa, Artificial Sequence
  kA2QA (Vk chain mutant)
AQVLTQTPSPVSAAVGGTVSISCQSSQSVHHNNYLAWFQQKSGQPPKQLIYAASTLAPGVPSRFKGSGSGTQFTLTISDVQCDDAATYYCAGLYNSGSDIYGFGGGTEVVVK

SEQ ID NO: 13 - protein, 112 aa, Artificial Sequence
  kS20T (Vk chain mutant)
AAVLTQTPSPVSAAVGGTVTISCQSSQSVHHNNYLAWFQQKSGQPPKQLIYAASTLAPGVPSRFKGSGSGTQFTLTISDVQCDDAATYYCAGLYNSGSDIYGFGGGTEVVVK

SEQ ID NO: 14 - protein, 112 aa, Artificial Sequence
  kA40S (Vk chain mutant)
AAVLTQTPSPVSAAVGGTVSISCQSSQSVHHNNYLSWFQQKSGQPPKQLIYAASTLAPGVPSRFKGSGSGTQFTLTISDVQCDDAATYYCAGLYNSGSDIYGFGGGTEVVVK

SEQ ID NO: 15 - protein, 112 aa, Artificial Sequence
  S46P (Vk chain mutant)
AAVLTQTPSPVSAAVGGTVSISCQSSQSVHHNNYLAWFQQKPGQPPKQLIYAASTLAPGVPSRFKGSGSGTQFTLTISDVQCDDAATYYCAGLYNSGSDIYGFGGGTEVVVK

SEQ ID NO: 16 - protein, 112 aa, Artificial Sequence
  Q52L (Vk chain mutant)
AAVLTQTPSPVSAAVGGTVSISCQSSQSVHHNNYLAWFQQKSGQPPKLLIYAASTLAPGVPSRFKGSGSGTQFTLTISDVQCDDAATYYCAGLYNSGSDIYGFGGGTEVVVK

SEQ ID NO: 17 - protein, 112 aa, Artificial Sequence
  P69S (Vk chain mutant)
AAVLTQTPSPVSAAVGGTVSISCQSSQSVHHNNYLAWFQQKSGQPPKQLIYAASTLASGVPSRFKGSGSGTQFTLTISDVQCDDAATYYCAGLYNSGSDIYGFGGGTEVVVK

SEQ ID NO: 18 - protein, 10 aa, Artificial Sequence
  exemplary sequence derived from the J1 joining region
FGGGTEVVVK

SEQ ID NO: 19 - protein, 11 aa, Artificial Sequence
  exemplary sequence derived from joining regions J2, J4, and J6
WGPGTLVTVSS

(19) United States
(12) Patent Application Publication
(10) Pub. No.: US 2021/0221915 A1
(43) Pub. Date: Jul. 22, 2021

(54) UNIVERSAL OR NORMALIZED ANTIBODY FRAMEWORKS FOR IMPROVED FUNCTIONALITY AND MANUFACTURABILITY

(71) Applicant: Ventana Medical Systems, Inc., Tucson, AZ (US)

(72) Inventors: Fernando Jose Rebelo do Cuoto, Pleasanton, CA (US); Melody S. Lee, Foster City, CA (US)

(21) Appl. No.: 15/734,301

(22) PCT Filed: Jun. 4, 2019
(86) PCT No.: PCT/EP2019/064428

§ 371 Date: Dec. 2, 2020

Related U.S. Application Data
(60) Provisional application No. 62/682,795, filed on Jun. 8, 2018.

(51) Int. Cl. C07K 16/44

(52) U.S. Cl. CPC C07K 16/44; C07K 2317/92; C07K 2317/565; C07K 2317/567; C07K 2317/14; C07K 2317/76; C07K 2317/94

(57) ABSTRACT

The invention provides methods of designing and manufacturing universal or normalized sequence templates for rabbit monoclonal antibodies for diagnostic applications. The invention further provides for methods of optimizing desirable antibody properties, such as thermal stability, long-term stability, expression, deamination/oxidation, and/or aggregation. The invention further provides for universal or normalized rabbit monoclonal antibody templates or frameworks and antibodies derived therefrom.

### CROSS-REFERENCE TO RELATED APPLICATIONS

Priority is hereby claimed to U.S. Provisional patent Application No. 62/682,795, filed Jun. 8, 2018, the content of which is incorporated herein by reference in its entirety.

### REFERENCE TO SEQUENCE LISTING SUBMITTED AS A COMPLIANT ASCII TEXT FILE (.TXT)

A Sequence Listing in the form of an ASCII-compliant text file (entitled “P34814WO_Sequence_ST25”) created on Apr. 26, 2019, and 22104 bytes in size) is submitted concurrently with the instant application, and the entire contents of the Sequence Listing are incorporated herein by reference.

### FIELD

The invention provides methods of designing universal or normalized sequence templates for manufacturing rabbit monoclonal antibodies for diagnostic applications by modifying variable regions. The invention further provides for methods of optimizing desirable antibody properties, such as thermal stability, long-term stability, expression, deamination/oxidation, and/or aggregation, which can adversely affect diagnostic assays, without adversely affecting binding specificity to the target. The invention further provides for universal or normalized antibody templates or frameworks, and rabbit monoclonal antibodies derived therefrom.

### BACKGROUND

There is an ongoing need to develop methods of improving reproducible conjugation ratios, and for standardizing antibody framework regions for stability and developability for use in diagnostic assays such as immunohistochemistry.

In principle, antibody engineering methods can be used to standardize antibody templates, such as frameworks and constant regions, while also maintaining antibody specificity and affinity.

Antibody complementarity determining regions (“CDRs”) are usually not modified because they are the principal determinants of antibody specificity and affinity for the target antigen. However, in some cases, CDR sequences themselves may be a source of antibody instability.

In practice, most of the accumulated antibody engineering knowledge relates to human and murine antibodies. However, rabbit monoclonal antibodies are used in several diagnostics applications both as primary antigen-detection reagents and as secondary signal generation or amplification reagents.

Primary antibodies are used to select particular tumor markers with exquisite specificity, and secondary antibodies are used to generate a detection signal. Each antibody is unique with respect to specificity, affinity, production levels, stability, and suitability to conjugation. This uniqueness stems from the amino acid sequence and poses a challenge in diagnostic assay development and manufacturing standardization. The present inventors have utilized protein engineering to solve this problem and have focused their efforts particularly on standardizing antibody framework regions for stability and, more generally, antibody developability.

There are numerous advantages to using rabbit monoclonal antibodies. By using rabbit monoclonal antibodies, the problem of certain proteins being recognized as self-antigens in humans and mice is avoided. Furthermore, rabbit monoclonal antibodies have an improved immune response to small epitopes, recognize a more diverse range of epitopes, and have a better response to mouse antigens. Thus, rabbit monoclonal antibodies give better reactions to antigens than those from other hosts, such as mice.

Moreover, use of rabbit monoclonal antibodies results in more accurate immunohistochemistry results, which can be attributed to a rabbit's unique immune system. Rabbits can generate a larger range of high-affinity antibodies than can mice. Therefore, it is more likely to find a rabbit antibody that can function in a range of applications than it is to find a similarly suitable mouse antibody. Additionally, numerous smaller peptides that elicit a poor response in mice generate a favorable response in rabbits. For these reasons, rabbit monoclonal antibodies are becoming more preferred in research and clinical applications.

US2004/0086979, incorporated herein by reference, discloses a method for resurfacing a rabbit antibody, said method including: (a) identifying a surface-exposed amino acid of a framework region of a parent rabbit antibody that differs from an amino acid at a corresponding position of a non-rabbit antibody by comparing the amino acid sequence of said framework region of said parent rabbit antibody to the amino acid sequence of said framework region of said non-rabbit antibody; and (b) substituting said identified amino acid with an amino acid at said corresponding position of a non-rabbit antibody, to resurface said rabbit antibody.

US2005/0033031, incorporated herein by reference, discloses a method of humanizing a rabbit monoclonal antibody, said method including: (a) comparing an amino acid sequences of a heavy an a light chain variable domain of a parent rabbit antibody to the amino acid sequence of a heavy and a light chain variable domain of a similar human antibody; and (b) altering amino acids within the framework regions of said heavy and light chain variable domains of said rabbit antibody such that the altered framework regions are more similar in sequence to the equivalent framework regions of said similar human antibody; wherein said altered amino acids are not involved in complementarity determining region (CDR) contacts, interchain contacts, or are buried residues with substantially different side chains.

U.S. Pat. No. 7,462,697, incorporated herein by reference, discloses a method for humanizing a rabbit antibody, including: a) identifying a variation tolerant position in a parent rabbit antibody by comparing its amino acid sequence to the amino acid sequences of a plurality of related antibodies that are obtained from the same rabbit as said parent rabbit antibody, in which said parent antibody and said related antibodies: i.) bind to the same antigen; ii.) each comprise heavy chain variable domains that have an overall amino acid sequence identity of at least 90% relative to one another; iii. each comprise light chain variable domains that have an overall amino acid sequence identity of at least 90% relative to one another; iv. have H3 CDRs that are identical in length and identical in sequence except for 0, 1 or 2 amino acid substitutions relative to one another; v. have L3 CDRs that are identical in length and identical in sequence except for 0, 1 or 2 amino acid substitutions relative to one another; b) aligning the amino acid sequence of said parent rabbit antibody with the amino acid sequence of a human antibody, in which said human antibody is one of ten human antibodies that are most homologous to said parent rabbit antibody; and c) substituting the amino acid present at said variation tolerant position with the corresponding amino acid of said human antibody to produce a humanized antibody.

Accordingly, there is a need to provide a standardized, universal or normalized (used interchangeably) rabbit monoclonal antibody framework or template that optimizes desirable antibody characteristics, such as thermal stability. Increased thermal stability and decreased aggregation leads to increased shelf-life, which is important for standardizing diagnostic assays.

### SUMMARY

The present invention provides for methods of designing and manufacturing a normalized rabbit antibody comprising selecting a wild-type rabbit antibody, and mutating said wild-type rabbit antibody to obtain a normalized rabbit antibody in order to optimize desirable properties.

In one aspect of the present disclosure is a method of making a universal or normalized rabbit antibody comprising selecting a wild-type rabbit antibody, comparing the wild-type antibody sequence to a normalized antibody sequence comprising a set of amino acid residues in the variable framework and/or joining regions that are most frequently-occurring at the same position in rabbit antibodies known to be thermally stable and/or having a long-term stability of at least 12 months, and identifying one or more amino acid residues in the wild type antibody that differ from the normalized sequence, and mutating said one or more amino acid residues of the wild type antibody identified in step (b) to the corresponding amino acid residue of the normalized sequence to obtain a universal or normalized rabbit antibody, wherein the binding affinity of the antibody to its target remains within an acceptable range when compared to the binding affinity of the unmodified wild-type antibody. In some embodiments, the universal or normalized rabbit antibody comprises an amino acid sequence according to SEQ ID NO: 1, 4, 5, 6, 7, 8, 9, or 10 in the VH chain. In some embodiments, the universal or normalized rabbit antibody comprises an amino acid sequence according to SEQ ID NO: 2, 12, 13, 14, 15, 16, or 17 in the Vk chain. In some embodiments, the universal or normalized rabbit antibody comprises an amino acid sequence according to SEQ ID NO: 1, 4, 5, 6, 7, 8, 9, or 10, and an amino acid sequence according to SEQ ID NO: 2, 12, 13, 14, 15, 16, or 17. In some embodiments, the universal or normalized rabbit antibody exhibits improved thermal stability as compared to the wild-type rabbit antibody. In some embodiments, the universal or normalized rabbit antibody exhibits improved long-term stability as compared to the wild-type rabbit antibody. In some embodiments, the universal or normalized rabbit antibody exhibits improved deamination as compared to the wild-type rabbit antibody. In some embodiments, the universal or normalized rabbit antibody exhibits improved expression as compared to the wild-type rabbit antibody. In some embodiments, the universal or normalized rabbit antibody is a primary rabbit antibody. In some embodiments, the mutation is in the J1 region of the kappa chain. In some embodiments, the mutation is in the FR4 region of the kappa chain. In some embodiments, the mutation is in the J2, J4, and/or J6 regions of the heavy chain. In some embodiments, the binding affinity of the antibody to its target remains within an acceptable range when compared to the binding affinity of the unmodified wild-type antibody. In some embodiments, the mutation is in a complementarity determining region.

In one aspect, the invention features a universal or normalized rabbit antibody obtained by the methods of the present disclosure.

In one aspect of the present disclosure is a universal or normalized rabbit antibody comprising an amino acid sequence selected from the group consisting of SEQ ID NO: 1, 4, 5, 6, 7, 8, 9, and 10 in the VH region.

In one aspect of the present disclosure is a universal or normalized rabbit antibody comprising an amino acid sequence selected from the group consisting of SEQ ID NO: 2, 12, 13, 14, 15, 16, and 17 in the Vk region.

In one aspect of the present disclosure is a universal or normalized rabbit antibody comprising an amino acid sequence in the VH region that is selected from the group consisting of SEQ ID NO: 1, 4, 5, 6, 7, 8, 9, and 10 and an amino acid sequence in the Vk region that selected from the group consisting of SEQ ID NO: 2, 12, 13, 14, 15, 16, and 17.

In some embodiments of the disclosed method, the mutated universal or normalized antibody has improved properties for diagnostic use. In some embodiments, the improved properties are selected from the group consisting of thermal stability; long-term stability; decreased aggregation and decreased oxidation/deamidation. In some embodiments of the disclosed method, the mutations are not in the complementarity determining region.

In one aspect of the present disclosure is a method of improving one or more properties in an antibody for diagnostic use, comprising selecting a wild-type rabbit antibody that is deficient in one or more characteristics selected from the group consisting of recombinant expression, thermal stability, long-term stability, aggregation, and oxidation/deamidation, comparing the wild-type antibody sequence to a normalized antibody sequence comprising a set of amino acid residues in the variable framework and/or joining regions that are most frequently-occurring at the same position in a set of rabbit antibodies known to have the characteristic within an acceptable range, and identifying one or more amino acid residues in the wild type antibody that differ from the normalized sequence, and mutating said identified one or more amino acid residues of the wild type antibody to the corresponding amino acid residue of the normalized sequence, wherein the one or more characteristics are improved relative to the unmodified wild-type antibody.

In one aspect of the present disclosure is a method of manufacturing a recombinant rabbit monoclonal antibody comprising selecting a wild-type rabbit antibody, comparing the wild-type antibody sequence to a normalized antibody sequence comprising a set of amino acid residues in the variable framework and/or joining regions that are most frequently-occurring at the same position in a set of rabbit antibodies known to have a characteristic within an acceptable range selected from the group consisting of thermal stability, long-term stability, decreased aggregation and decreased oxidation/deamidation, and identifying one or more amino acid residues in the wild type antibody that differ from the normalized sequence, and preparing a standard expression vector encoding a complementarity determining region containing alterations to the wild-type sequence in one or more of the identified residues, expressing said vector in a suitable host cell, and purifying said antibody from step. In some embodiments, said set of amino acid residues of the normalized sequence are the most frequently-occurring at the same position in at least 50 rabbit antibodies having the relevant characteristic. In some embodiments, said set of amino acid residues of the normalized sequence are the most frequently-occurring at the same position in at least 100 rabbit antibodies having the relevant characteristic. In some embodiments, said set of amino acid residues of the normalized sequence are the most frequently-occurring at the same position in at least 150 rabbit antibodies having the relevant characteristic. In some embodiments, said set of amino acid residues of the normalized sequence are the most frequently-occurring at the same position in at least 200 rabbit antibodies having the relevant characteristic. In some embodiments, said set of amino acid residues of the normalized sequence are the most frequently-occurring at the same position in at least 250 rabbit antibodies having the relevant characteristic. In some embodiments, said set of amino acid residues of the normalized sequence are the most frequently-occurring at the same position in at least 300 rabbit antibodies having the relevant characteristic.

The present inventors have surprisingly found that antibodies obtained by the present invention exhibit superior properties without substantially affecting binding affinity and specificity to the target.

The disclosure further provides for normalized monoclonal rabbit antibodies obtained by selecting a wild-type rabbit antibody, and mutating said wild-type rabbit antibody.

Further objects, features, and advantages of the invention will become apparent from the detailed description that follows.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts (A) an overview of the protein engineering strategy employed according to the present invention, and (B) a schematic overview of methods, wherein a scripting program was used to generate variants based on the universal template. These variants for the light (pink) and heavy (purple) chains were gene synthesized and then cloned into either pTT5 or pDV3a vectors prior to transient transfection of HEK293 cells. Cultures were grown in a shake flask for a period of six days and antibody supernatant was harvested and purified as needed. Antibody supernatants were diluted serially to determine antibody concentration and used to determine binding affinity. Thermal stability of the antibody supernatants was tested by ELISA. Purified Ab was used in the melting and aggregation temperature measurements. Immunohistochemistry was performed on control tissues.

FIG. 2 depicts (A) the VH domain of a rabbit (yellow) and mouse (green) antibody, and (B) a close-up view of a Tyr corner in the VH domain.

FIG. 3 depicts the distribution of amino acids in rabbit antibody sequences.

FIG. 4 depicts universal or normalized antibody templates of the present invention. Lines: (NO) numbering system (mostly IMGT) and approximate locations of FRs and CDRs; (IK) important Vk positions; (TK) universal or normalized Vk template; (IH) important VH chain positions; (TH) universal or normalized VH template. IK and IH lines: (c) probable CDR contact; (d) buried and probable CDR contact; (2) buried polar region 2; (b) buried; (0) no amino-acid residue. (-) no preference; (i) probable interchain contact; (t) buried tyrosine corner; (v) turn; (x) surface residue that can be changed with very low risk of altering affinity; (u/c/b) vaguely defined CDR's, these residues can be conservatively changed albeit with a high risk of altering affinity; (s) either the first position after the signal peptide (a possible CDR contact) or residue VH84 in the D-E loop, which is absent in some rabbit VH chains.

FIG. 5 depicts (A) the VH and Vk series mutants and binding curves for (B) VH variants paired with WT Vk, and (C) Vk variants paired with WT VH, and (D) determinants of synthetic lethality.

FIG. 6 depicts (A) thermal stability curves for the wild-type (“WT”) Vk with VH mutants, (B) thermal stability curves for the WT VH and Vk mutants, and (C) thermal stability curves for xCD2 WT versus the most mutated VH and Vk.

FIG. 7 depicts (A) the effect of conversion to the universal or normalized template on expression levels, melting point, and temperatures of aggregation (“T_{agg}266” and “T_{agg}473”), and (B) the similar staining patterns of WT xCD2 antibody and xCD2 antibody converted to the universal or normalized template of the present invention.

### ABBREVIATIONS

“FR” refers to “framework region.”
“CDR” refers to “complementarity determining region.”
“V” refers to variable region.
“Vk” refers to variable kappa chain (light chain).
“Vλ.” refers to variable lambda chain (light chain).
“VH” refers to variable heavy chain.
“J” refers to “joining region.”

### DETAILED DESCRIPTION

In order to facilitate review of the various embodiments of the disclosure, the following explanations of specific terms are provided:

Antibody: A polypeptide that includes at least a light chain or heavy chain immunoglobulin variable region and specifically binds an epitope of an antigen. Antibodies include monoclonal antibodies, polyclonal antibodies, or fragments of antibodies as well as others known in the art. In some examples, an antibody is linked or conjugated to another molecule, such as a nanoparticle (for example, a gold nanoparticle) or an enzyme (for example, alkaline phosphatase).

Antibodies are composed of a heavy and a light chain, each of which has a variable region, termed the variable heavy (VH) region and the variable light (VL) region. Together, the VH region and the VL region are responsible for binding the antigen recognized by the antibody. This includes intact immunoglobulins and the variants and portions of them well known in the art, such as Fab′ fragments, F(ab)′2 fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes recombinant forms such as chimeric antibodies (for example, humanized murine antibodies) and heteroconjugate antibodies (such as, bispecific antibodies). See also, Pierce Catalog and Handbook, 1994-1995 (Pierce Chemical Co., Rockford, Ill.); Kuby, Immunology, 3rd Ed., W.H. Freeman & Co., New York, 1997.

A “monoclonal antibody” is an antibody produced by a single clone of B lymphocytes from mouse or rabbit or by a cell, e.g., HEK293 cell, into which the light and heavy chain genes of a single antibody have been transfected. Monoclonal antibodies are produced by methods known to those of ordinary skill in the art, for instance by making hybrid antibody-forming cells from a fusion of myeloma cells with immune spleen cells. These fused cells and their progeny are termed “hybridomas.” Monoclonal antibodies include humanized monoclonal antibodies.

Primary (1°) antibodies are antibodies that are immunospecific for one or more components of a biological sample. They are frequently used in diagnostic applications requiring detection and analysis of biomarkers in a sample, for example, immunohistochemistry, immunocytochemistry, flow cytometry, and the like. Secondary (2°) antibodies are antibodies that are immunospecific for another antibody (or a component of the other antibody), and are frequently used in diagnostic applications relying on indirect detection of biomarkers of interest. For example, in immunohistochemical and immunocytochemical applications, a primary antibody is used to mediate deposition of a detectable moiety on a histological or cytological sample in close proximity to the biomarker to which the primary antibody is bound. In some cases, the detectable moiety is directly conjugated to the primary antibody, and thus is deposited on the sample upon binding of the biomarker-specific reagent to its target (generally referred to as a direct labeling method). In other embodiments, deposition of the detectable moiety is effected by binding a secondary antibody to the primary antibody bound to the sample, followed by a set of detection reagents that bind to or otherwise react with the secondary antibody in a manner that effects deposition of the detectable moiety (generally referred to as an indirect labeling method). Non-limiting examples of commercially available reagents or kits for use in indirect histochemical or cytochemical detection methods include: VENTANA ultraView detection systems (secondary antibodies conjugated to enzymes, including HRP and AP); VENTANA iVIEW detection systems (biotinylated anti-species secondary antibodies and streptavidin-conjugated enzymes); VENTANA OptiView detection systems (OptiView) (anti-species secondary antibody conjugated to a hapten and an anti-hapten tertiary antibody conjugated to an enzyme multimer); VENTANA Amplification kit (unconjugated secondary antibodies, which can be used with any of the foregoing VENTANA detection systems to amplify the number of enzymes deposited at the site of primary antibody binding); VENTANA OptiView Amplification system (Anti-species secondary antibody conjugated to a hapten, an anti-hapten tertiary antibody conjugated to an enzyme multimer, and a tyramide conjugated to the same hapten. In use, the secondary antibody is contacted with the sample to effect binding to the primary antibody. Then the sample is incubated with the anti-hapten antibody to effect association of the enzyme to the secondary antibody. The sample is then incubated with the tyramide to effect deposition of additional hapten molecules. The sample is then incubated again with the anti-hapten antibody to effect deposition of additional enzyme molecules. The sample is then incubated with the detectable moiety to effect dye deposition); VENTANA DISCOVERY, DISCOVERY OmniMap, DISCOVERY UltraMap anti-hapten antibody, secondary antibody, chromogen, fluorophore, and dye kits, each of which are available from Ventana Medical Systems, Inc. (Tucson, Ariz.); PowerVision and PowerVision+ IHC Detection Systems (secondary antibodies directly polymerized with HRP or AP into compact polymers bearing a high ratio of enzymes to antibodies); and DAKO EnVision™+ System (enzyme labeled polymer that is conjugated to secondary antibodies).

Regardless of the type of antibody or detection system used, each Ab is unique with respect to specificity, affinity, production levels, stability, and suitability to conjugation, which poses a challenge in assay development and manufacturing standardization. The present inventors use protein engineering to solve this problem and have focused on a) cloning of hybridomas for conversion into recombinant antibodies, b) modifying antibody sequences to accommodate higher and more reproducible conjugation ratios, and c) standardizing antibody framework regions for stability and, more generally, antibody developability.

The present inventors have surprisingly found that methods of the present invention can be used to manufacture normalized antibodies for improved reproducibility and development.

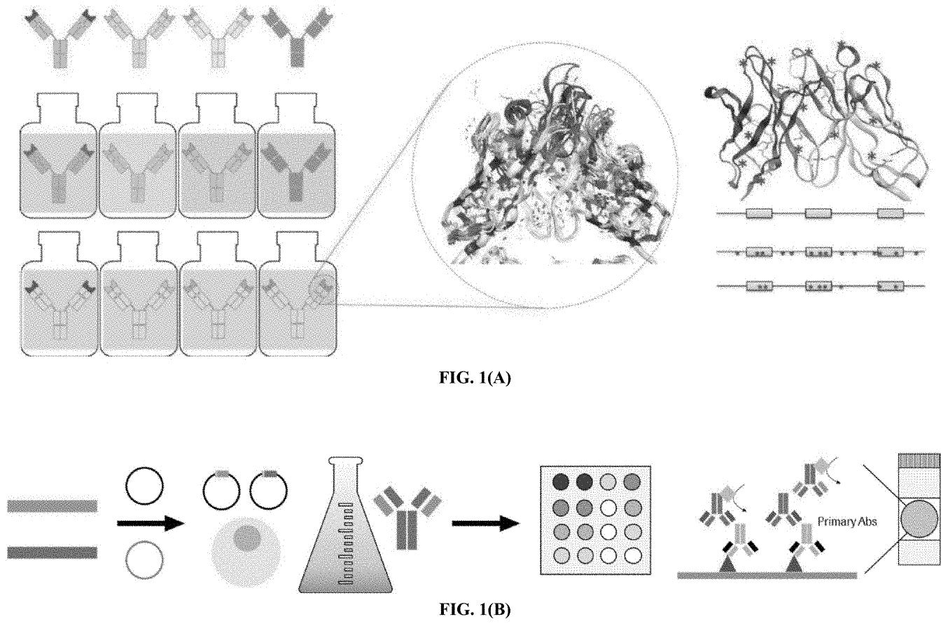

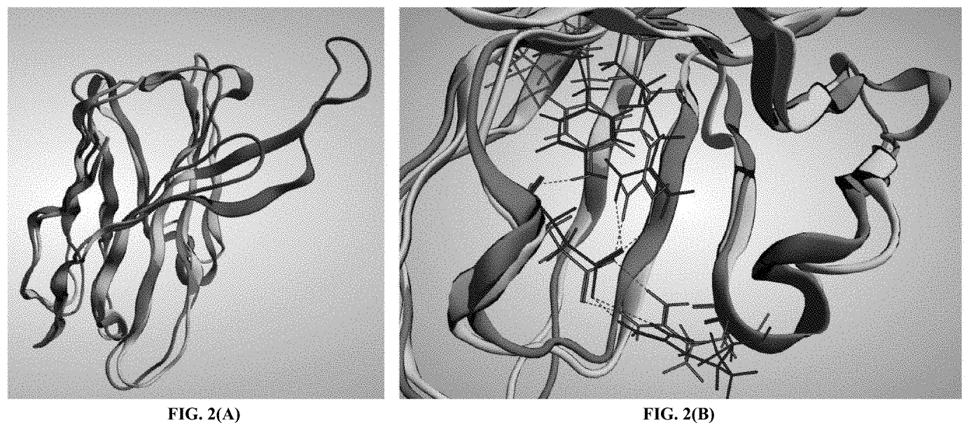

FIG. 1A shows an overview of an exemplary protein engineering strategy. Hybridomas can lose antibody production over time, therefore, conversion to a recombinant version of the antibody may be necessary for certain clones. Recombinant antibodies can also enable protein engineering to introduce site-specific conjugation for reliable labelling and to optimize antibody composition for developability, stability, and other desired properties required for downstream applications. Current methods for conjugation rely on the natural properties available in the antibody, thus producing random distribution of bioconjugates. Introducing site-specific conjugation sites can greatly improve manufacturability and product composition. With the engineered recombinant antibody in hand, the antibody clone may be eventually introduced into CHO cell-based targeted integration system. This may allow for large scale expression, production, and manufacturing that is on par with the amount of antibody produced by the hybridoma.

FIG. 1B shows existing antibody vectors or cDNA from hybridoma clones may be used as starting templates from which light chains (pink) and heavy chains (purple) are amplified using specifically designed primers and polymerase chain reaction. The antibody chains are cloned into either pTT5 or pDV3a vectors prior to transient transfection of HEK293 cells, and cultures are grown in a shake flask for a period of six days. Antibody supernatant is harvested and purified, and purified antibodies are characterized with ELISA assays to determine antigen binding and immunohistochemistry to verify function.

The present inventors generated a “universal or normalized stable scaffold template” and presented its application towards the stabilization of a rabbit monoclonal Ab. Specifically, the present inventors have found that a) there is no need to prolong the use of hybridomas and risk loss of specific antibody expression due to hybridoma instability, and b) variable regions of antibodies can be re-engineered to introduce desirable formulation properties such as stability without loss of specificity and affinity.

Although in principle engineering can be used to standardize antibody scaffolds while maintaining antibody specificity and affinity, in practice most of the accumulated antibody engineering knowledge relates to human and murine antibodies. The present inventors examined the structure and sequence of rabbit antibodies and found many similarities with human and murine antibodies and created a slightly modified IMGT numbering system for rabbit variable heavy (VH) and variable kappa light (Vk) chains to generate a structural alignment. The most frequently occurring amino acids (Aa's) in the framework (FR) sequence may lead to higher stability. Therefore, for positions that are not important for binding, frequently occurring Aa's or those present in rabbit antibodies with no known stability issues may be used to build a universal or normalized antibody scaffold that may act as a template to convert unstable antibodies.

The present inventors investigated germline-derived and frequently occurring amino acid sequences in rabbit antibodies, with the view that these sequences would lead to higher stability and other desirable properties. For sequence positions where there was no strong bias, the present inventors used frequently occurring amino acids. Additionally, the present inventors used amino acids present in antibodies with no known stability issues. Loosely defined CDRs may be generally left intact.

The present inventors first compared the sequence and structure of rabbit Vk and VH regions with their human and mouse counterparts and verified that individual rabbit antibody amino-acid residues have similar structural roles as their corresponding human and mouse counterparts.

The present inventors first compared the sequence and structure of rabbit Vk and VH regions with their human and mouse counterparts.

Antibodies according to the present invention are manufactured from one or more universal or normalized antibody templates covering the frameworks of kappa and heavy variable regions (Vk, Vk, and VH, respectively). Framework regions are found between the hypervariable regions in variable chains of antibodies. Framework regions form a beta-sheet (beta-barrel) structure which serves as a scaffold to hold the hypervariable regions in position to contact antigens or targets.

These universal or normalized antibody templates of the present invention were generated on the basis of structural and sequence analyses of variable regions of existing rabbit monoclonal antibodies.

In one embodiment, the present disclosure provides for methods by which the skilled artisan can change as many residues as possible from a candidate antibody to the corresponding residues in the universal or normalized template to improve desired properties with no considerable loss of binding affinity. In another embodiment, methods according to the present invention are applicable to CDR sequences whereby stability is increased without loss of affinity.

In one embodiment, binding affinity is determined by assessing the dissociation constant (K_{D}) of the binding of the antibody to the target. K_{D} of a binding pair can be assessed using surface-based (heterogeneous) methods including surface plasmon resonance (SPR), biolayer interferometry (BLI) and enzyme linked immunosorbent assays (ELISA). Schuck 1997; Gauglitz 2008; Friguet et al. 1985.

Generally, the K_{D} of the universal or normalized antibodies will be in the range 10⁻⁶ to 10⁻¹² (M), but in any case, and may be improved relative to the wild-type antibody.

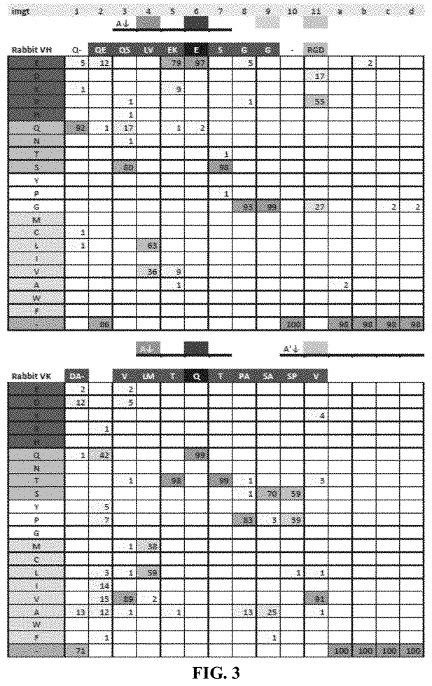

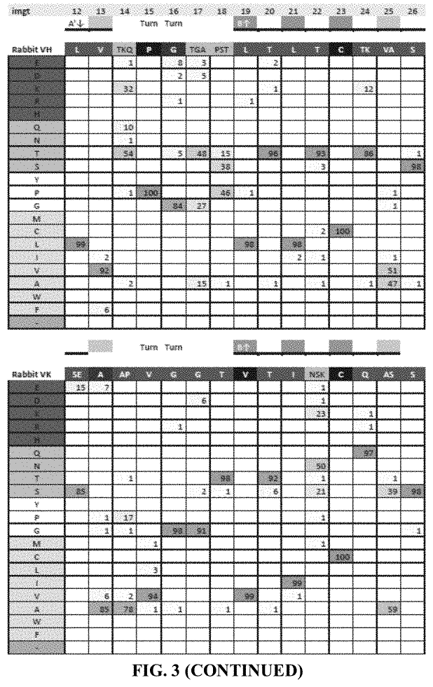

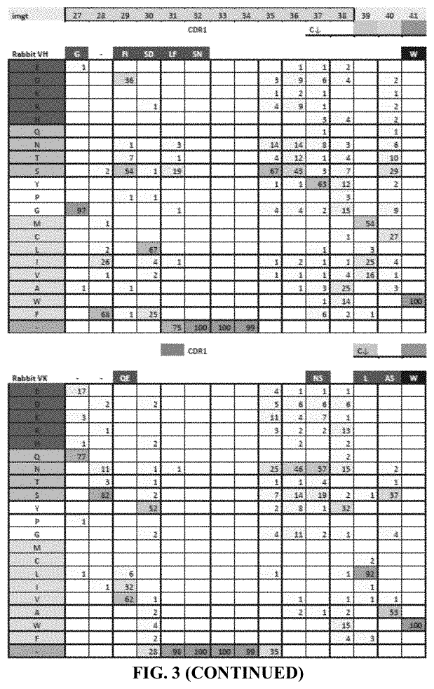

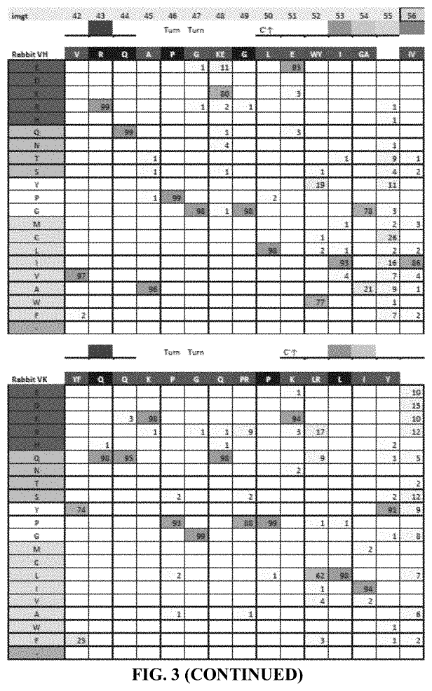

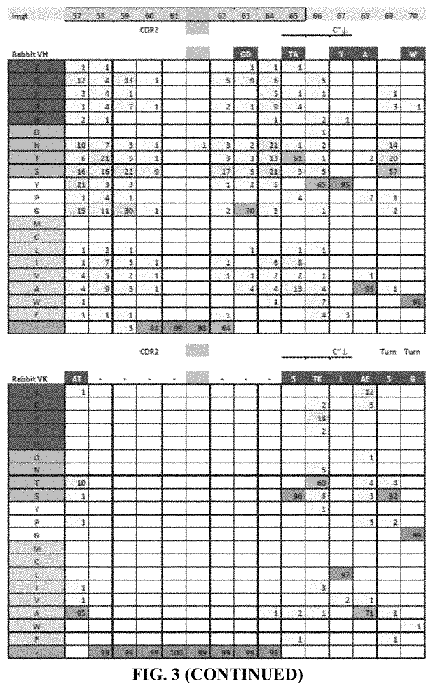

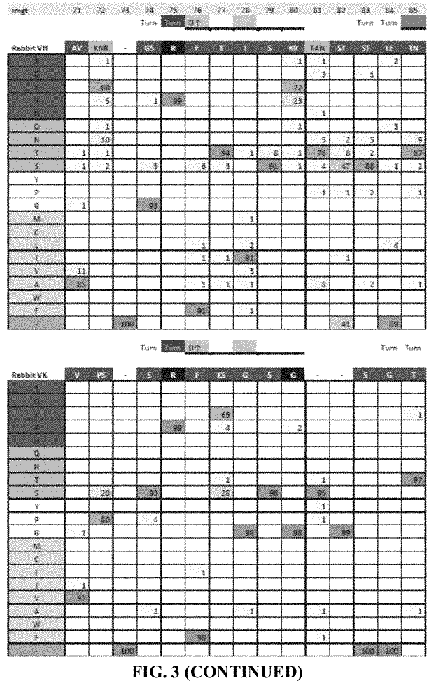

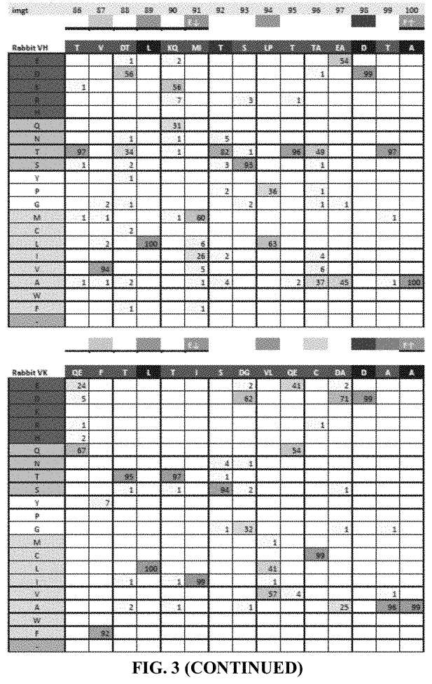

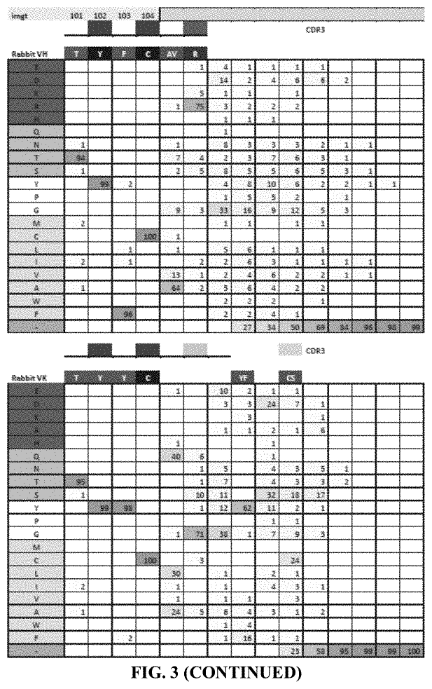

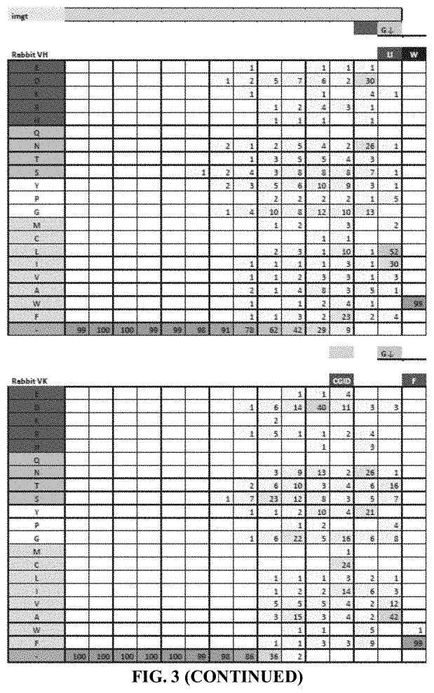

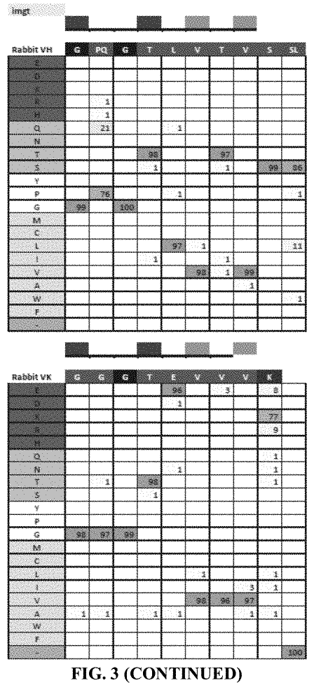

The present inventors collected approximately 300 amino acid sequences from antibodies against various antigens and peptides from antibodies independently isolated by Spring Bioscience. The present inventors further collected nine (9) VH/Vk sequences of published rabbit antibody structures: 4HBC, 4HT1, 4JO1, 4JO4, 4MA3, 4O4Y, 4ZTP, SCON, SDRN, which were downloaded from the RCSB Protein Databank. The distribution of amino acids in these sequences is depicted in FIG. 3, wherein the intensity of the green coloring is proportional to the percent incidence of amino acids at the indicated positions.

The present inventors further collected 39 VH and 65 Vk1 functional germline sequences (not analyzed in FIG. 3).

VH and Vk sequences were then aligned using the mostly the IMGT numbering system (see Lefranc et al., 2003, “IMGT unique numbering for immunoglobulin and T cell receptor variable domains and Ig superfamily V-like domains”) and structural alignments published for mouse and human antibodies (see Honegger and Pluckthun, 2001, “Yet another numbering scheme for immunoglobulin variable domains: an automatic modelling and analysis tool”).

All framework amino acid positions and position-dependent structural properties generally applicable to all human, murine, and rabbit antibodies were annotated and verified as previously described (Chothia et al. 1998; Narciso et al. 2011), with the exceptions of a) the frequent presence of additional disulfide bonds formed between VH FR2 C40 and C55, b) internal CDR3 disulfide bonds in a few Vk1 sequences, c) an interdomain disulfide bond between a Cys residue in Vk FR3 (kabbat C80) or in FR4 (last position) and a Cys residue in CK, and d) a shortened DE loop in many rabbit VH FR3 regions, which can lack residue VH85 or both residues VH84 and VH85.

Using these numbering systems together, the present inventors then calculated the occurrence frequencies for each of the twenty standard amino acid residues at each of the VH and Vk structure-aligned positions. The proposed universal or normalized Vk and VH templates are shown in FIG. 4.

For the kappa chain framework 4 (“FR4”), an exemplary sequence of FGGGTEVVVK (SEQ ID NO: 18) was selected, which is derived from the J1 joining region.

For the heavy chain, an exemplary sequence of WGPGTLVTVSS (SEQ ID NO: 19) was selected, which is derived from joining regions J2, J4, and J6.

These Vk and VH FR4 sequences are found in most cloned antibodies with no known stability problems.

The employed numbering system is also depicted in FIG. 4, which applies to both the VH and Vk chains. Dark blue coloring indicates 100% conservation in the germline. For FR4, the sequences correspond respectively to rabbit germlines KJ1 and HJ2/4/6. Dark green coloring indicates 89% occurrence in antibodies provided by Spring Bioscience. Light green coloring indicates residues commonly found in antibodies with no known stability problems. Finally, “-” indicates an amino acid position that should be preserved as in the original wild type sequence.

In an embodiment, the recombinant universal or normalized antibodies of the present invention are produced by any host species suitable for the production of diagnostic antibodies. In one embodiment, the host cells are mammalian cells. In a specific embodiment, the host cells are HEK293F cells. In another specific embodiment, the host cells are CHO cells. In further embodiments, the host cells are yeast, bacterial (e.g., E. coli) or filamentous fungal cells. In a further embodiment, the host cells are insect cells. Purification of the recombinant universal or normalized antibody may be achieved using any known method, including chromatography and affinity chromatography.

In another embodiment, antibodies of the present invention are rabbit, mouse, or human antibodies

In another embodiment, antibodies of the present invention are rabbit antibodies.

In an embodiment, antibodies of the present invention are primary rabbit antibodies.

In an embodiment, antibodies of the present invention are secondary rabbit antibodies.

In an embodiment, antibodies of the present invention are humanizable.

In another embodiment, the antibodies are antibody fragments or single-chain antibodies including Fab and single chain Fv (scFv).

In another embodiment, the antibodies are chimeric antibodies having constant regions from a different antibody and/or species.

In an embodiment, the antibodies of the present invention are of any isotype including IgA, IgM, IgG (all sub-types), IgA and IgE.

In an embodiment, antibodies of the present invention exhibit improved thermal stability, long-term stability, recombinant expression/titers, deamination/oxidation, and/or aggregation relative to the wild-type, unmodified antibody.

Stability assays known in the art may be used to measure thermal stability/denaturation. In one embodiment, immunohistochemistry may be used. In another embodiment, ELISA may be used. In another embodiment, 2D electrophoresis is used. In another embodiment, differential scanning calorimetry and/or circular dichroism spectroscopy may be used. In a further embodiment, nano-differential scanning fluorimetry can be used. See, e.g., Vermeer and Norde, 2000; Svilenov et al. 2018; Strutz 2016, Amin et al. 2014. In one embodiment, increased melting temperature correlates with improved thermal stability.

Protein aggregation can be measured using a variety of known techniques including mass spectrometry, size exclusion chromatography (SEC), dynamic light scattering (DLS), light obscuration (LO), dynamic imaging particle analysis (DIPA) techniques such as micro-flow imaging (MFI), and Coulter counter (CC). Engelsman et al., Strategies for the Assessment of Protein Aggregates in Pharmaceutical Biotech Product Development; Pharm. Res. 2011; 28(4): 920-33.

In an embodiment, an antibody of the present invention exhibits improved thermal stability.

In another embodiment, an antibody of the present invention exhibits improved long-term stability. Long-term stability includes at least 6 months, preferably at least 1 year, more preferably at least two years and most preferably at least three years at a temperature of between −20 and −70° C. In one embodiment, the antibody is lyophilized.

In another embodiment, an antibody of the present invention exhibits improved expression, as determined by e.g., increased titers from recombinant expression compared to a corresponding unmodified wild-type antibody. In one embodiment, the titers are at least 0.5 g/L, more preferably at least 0.75 g/L. In another embodiment, increased expression could be measured by taking an OD280 reading. In yet other embodiments, surface plasmon resonance (SPR) can also be used to measure concentration and protein gel electrophoresis with standards could also be used to determine antibody concentration.

In another embodiment, an antibody of the present invention exhibits improved deamination/oxidation (i.e., less deamidation/oxidation). Deamidation is a spontaneous reaction that occurs when the side chains of asparagine and glutamine are modified and can result in cyclic intermediate compounds. Oxidation occurs primarily on cysteine and methionine, the sulfur-containing residues, resulting in the formation of intra-chain or inter-chain disulfide bonds. Methods of assessing oxidation are described in detail in Yan. 2009. Tryptophan is also oxidized easily. Tryptophan loss could be elucidated by the identification Trp degradation compounds including oxindolylalanine (Oia), 3a-hydroxy-1,2,3,3a,8,8a-hexahydropyrrolo[2,3-b]indole-2-carboxylic acid (PIC), N-formylkynurenine (NFK), dioxindolylalanine (DiOia), kynurenine (Kyn), and 5-hydroxytryptophan (5-OH-Trp).

In another embodiment, an antibody of the present invention exhibits improved aggregation (i.e., less aggregation).

In an embodiment, an antibody of the present invention comprises an amino acid sequence according to SEQ ID NO:1, 4, 5, 6, 7, 8, 9, or 10, which set forth a universal or normalized or normalized Vk template (SEQ ID NO: 1) and mutant variants thereof.

In an embodiment, an antibody of the present invention comprises an amino acid sequence according to SEQ ID NO: 2, 12, 13, 14, 15, 16, or 17, which set forth a universal or normalized VH template (SEQ ID NO: 2) and mutant variants thereof.

In an embodiment, an antibody of the present invention comprises an amino acid sequence according to SEQ ID NO:1, 4, 5, 6, 7, 8, 9, or 10, and an amino acid sequence according to SEQ ID NO: 2, 12, 13, 14, 15, 16, or 17.

In an embodiment, an antibody of the present invention comprises the amino acid sequence according to SEQ ID NO1.

In an embodiment, an antibody of the present invention comprises the amino acid sequence according to SEQ ID NO:2.

In an embodiment, an antibody of the present invention comprises the amino acid sequences according to SEQ ID NO:1: and SEQ ID NO:2.

The foregoing amino acid sequences as disclosed herein can be further modified by the substitution of one or more residues. Such substitutions may be of a conservative nature, for example, where one amino acid is replaced by an amino acid of similar structure and characteristics, such as where a hydrophobic amino acid is replaced by another hydrophobic amino acid.

Even more conservative would be replacement of amino acids of the same or similar size and chemical nature, such as where leucine is replaced by isoleucine. In studies of sequence variations in families of naturally occurring homologous proteins, certain amino acid substitutions are more often tolerated than others, and these are often show correlation with similarities in size, charge, polarity, and hydrophobicity between the original amino acid and its replacement, and such is the basis for defining “conservative substitutions.”

Conservative substitutions can be exchanges within one of the following five groups: Group 1-small aliphatic, nonpolar or slightly polar residues (Ala, Ser, Thr, Pro, Gly); Group 2-polar, negatively charged residues and their amides (Asp, Asn, Glu, Gln); Group 3-polar, positively charged residues (His, Arg, Lys); Group 4-large, aliphatic, nonpolar residues (Met, Leu, Ile, Val, Cys); and Group 5-large, aromatic residues (Phe, Tyr, Trp).

Less conservative substitutions might involve the replacement of one amino acid by another that has similar characteristics but is somewhat different in size, such as replacement of an alanine by an isoleucine residue.

Highly non-conservative replacements might involve substituting an acidic amino acid for one that is polar, or even for one that is basic in character. Such “radical” substitutions cannot, however, be dismissed as potentially ineffective since chemical effects are not totally predictable and radical substitutions might well give rise to serendipitous effects not otherwise predictable from simple chemical principles.

In one embodiment, the invention includes an amino acid sequence having at least 70%, at least 75%, at least 80%, at least 85%, at least 90%, or at least 95% percent identity to any of SEQ ID NO:1, 4, 5, 6, 7, 8, 9, or 10, or SEQ ID NO: 2, 12, 13, 14, 15, 16, 17, 18 or 19. The percent identity may be determined, for example, by comparing sequence information using the GAP computer program, version 6.0 described by Devereux et al. 1984 and available from the University of Wisconsin Genetics Computer Group (UWGCG). The GAP program utilizes the alignment method of Neddleman and Wunsch 1970, as revised by Smith and Waterman 1981. In another embodiment, the invention includes an amino acid sequence having at least 70%, at least 75%, at least 80%, at least 85%, at least 90%, or at least 95% percent identity to any of SEQ ID NO:1, 4, 5, 6, 7, 8, 9, or 10, or SEQ ID NO: 2, 12, 13, 14, 15, 16, 17, 18 or 19, and exhibits improved properties such as thermal stability, long-term stability, and expression.

In a specific embodiment, the universal or normalized or normalized antibody contains a wild-type Vk chain and a VH chain having at least one mutation.

In a specific embodiment, the universal or normalized or normalized antibody contains a wild-type Vk chain and a VH chain having the Y52W mutation.

In another specific embodiment, the universal or normalized or normalized antibody contains a wild-type Vk chain and a VH chain with all the following mutations: V4L; _83S; S96T; A54G; N101T; and Y52W, wherein indicates an amino acid position that should be preserved as in the original wild type sequence. In yet another specific embodiment, the universal or normalized or normalized 1 antibody contains a wild-type Vk chain and a VH chain with all the following mutations: V4L; R48K; _83S; S96T; A54G; and N101T.

In a specific embodiment, the universal or normalized antibody comprises a Vk chain having only one mutation and a wild type VH chain. In another embodiment, the universal or normalized or normalized antibody contains a wild-type Vk or Vλ, chain and a mutated VH chain. In another specific embodiment, the universal or normalized antibody contains a wild-type Vk or Vλ, chain and a VH chain having one or more of the following mutations: V4L; R48K; _83S; S96T; A54G, N101T; or Y52W.

In another embodiment, the universal or normalized antibody comprises a Vk or Vλ, chain having at least one mutation and a VH chain having at least one mutation. In a specific embodiment, the Vk chain mutation is A2Q; S20T; S46P,

A40S, Q52L or P69S. In another specific embodiment, the Vk chain comprises at least two of the foregoing mutations with the proviso that the mutation is not both S20T and Q52L. In another specific embodiment, the Vk chain comprises all the following mutations: A2Q, 520T, S46P, A40S and P69S. In another specific embodiment, the Vk chain comprises all of the following mutations A2Q; S46P, A40S, Q52L and P69S.

In another embodiment, the universal or normalized antibody comprises a Vk chain having one or more of the following mutations: A2Q; 520T; A40S, S46P, Q52L or P69S, with the proviso that the mutation is not both S20T and Q52L; and the VH chain has one or more of the following mutations: V4L; R48K, _83S; S96T; A54G, N101T; and Y52W.

In another embodiment, the universal or normalized antibody comprises a Vk or V [symbol] , chain having at least one mutation and a VH chain with all the following mutations: V4L; R48K, _83S; S96T; A54G, N101T; and Y52W.

In another embodiment, the universal or normalized antibody is used in a diagnostic assay. In one embodiment, the assay is immunohistochemistry (IHC). In another embodiment, the assay is enzyme-linked immunosorbent assay (ELISA). In another embodiment, the diagnostic assay is immunocytochemistry (ICC). In a further embodiment, the diagnostic assay is flow cytometry or FACS. In yet a further embodiment, the diagnostic assay is radioimmunoassay (MA).

The methods of the present invention are not limited to use on antibodies known or discovered to have problems with stability or aggregation or the other properties address above. In another embodiment, the methods of the present invention can be used to standardize panels of antibodies for immunoassays.

The following examples serve to illustrate certain embodiments of the disclosure and are not intended to limit the disclosure. Indeed, the same method can be used to modify other rabbit antibodies.

### EXAMPLES

### Example 1—Mutagenesis for Conversion to Universal or Normalized Template

A rabbit monoclonal antibody raised by Spring Bioscience was selected against a 9-mer peptide. a T_{M} of 73.1C,T_{agg}266 of 73.4C T_{agg}473 of 73.8C and an expression yield of 0.51 mg/ml.

The selected rabbit monoclonal antibody's Vk and VH-encoding DNA sequences were obtained from Spring Bioscience and expression vectors were constructed.

The expression vectors were based on the pDV2/pRK-Fc system ((Shang et al. 2015; Tesar and Hötzel 2013) modified to express a rabbit IgG constant region instead of the original human sequence.

Concentrations were determined by comparing absorbance values of sample antibodies determined at several dilutions with those of standard curve.

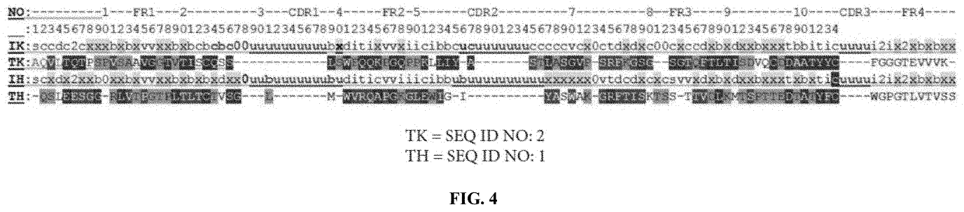

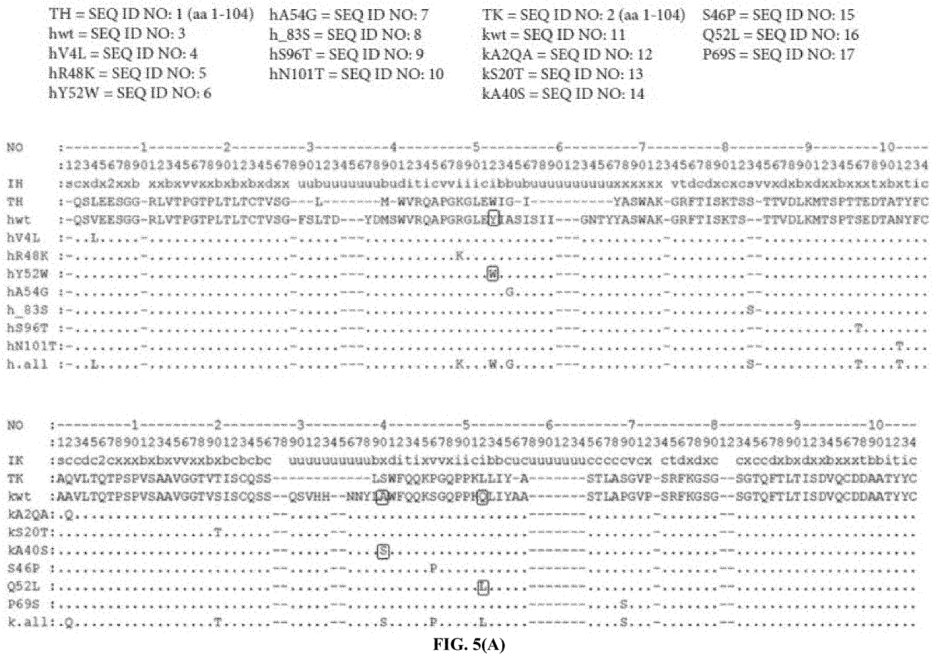

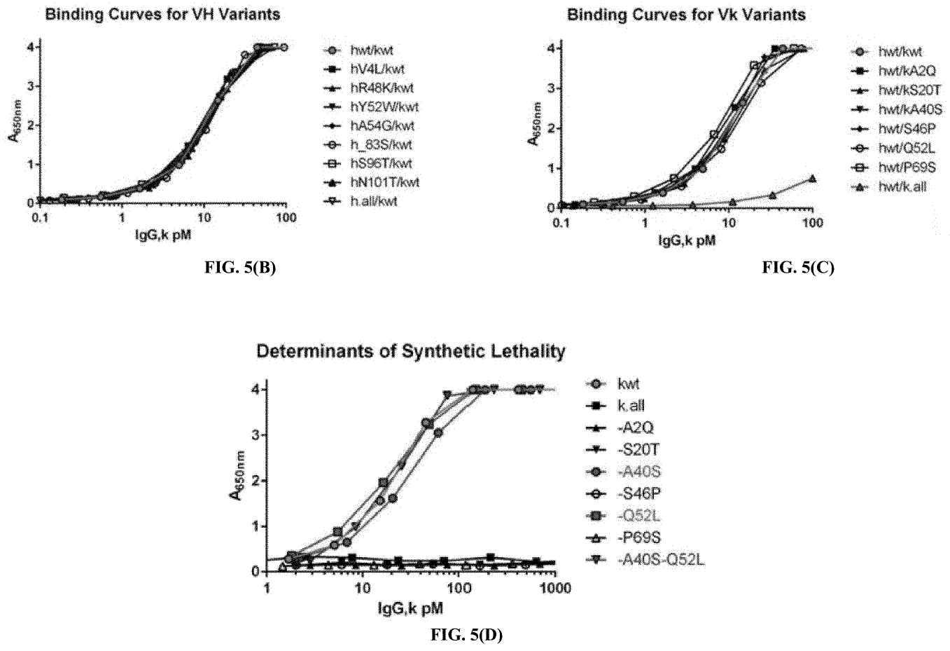

A series of VH and Vk chains were synthesized with both individual and multiple sequence changes from the original wild-type antibody to the universal or normalized template (see FIG. 5A, which depicts the VH and Vk series).

CDR3 and FR4 regions, past IMGT residue C104, were kept as in the wild-type antibody. The wild-type FR4 sequences matched the universal or normalized template's exactly. FR4 is identical to the universal or normalized templates. (NO, IM, TH) as in FIG. 5. VH variants: (hwt) WT HC. Vk variants: (kwt) wt light chain.

The VH and Vk series were expressed in HEK293F cells such that each mutated heavy chain was combined with the wild-type kappa chain, and vice-versa.

ELISA was used to measure rabbit antibody concentrations and to compare antibody binding between all variants as described below.

### Example 2—ELISA Assays

Antibody concentrations and EC50 were determined via ELISA.

Concentrations were determined by comparing absorbance values of sample antibodies determined at several dilutions with those of standard curve.

Gene synthesis was used to generate different VH and Vk single and combination mutants before cloning into pTT5 or pDV3a vectors. CDR3 and FR4 were left unchanged and FR4 was identical to the universal or normalized template.

Table 1 sets forth the wild-type and mutant sequences assayed:

TABLE 1
Wild-Type and Mutant
VH and Vk Sequences

| SEQ | Wild-type/universal |
|---|---|
| ID | or normalized |
| NO: | Template/Mutant |
| 1 | TH/h.all (universal |
| | or normalized VH |
| | template) |
| | (V4L; R48K, _83S; |
| | S96T; A54G, N101T; |
| | and Y52W) |
| 2 | TK/k.all (universal |
| | or normalized Vk |
| | template) |
| | (A2Q; S20T; S46P, |
| | Q52L and P69S) |
| 3 | hwt (wild-type VH chain) |
| 4 | hV4L (VH chain mutant) |
| 5 | hR48k (VH chain mutant) |
| 6 | hY52W (VH chain mutant) |
| 7 | hA54G (VH chain mutant) |
| 8 | h_83S (VH chain mutant) |
| 9 | hS96T (VH chain mutant) |
| 10 | hN101T (VH chain mutant) |
| 11 | kwt (wild-type Vk chain) |
| 12 | kA2QA (Vk chain mutant) |
| 13 | kS20T (Vk chain mutant) |
| 14 | kA40S (Vk chain mutant) |
| 15 | S46P (Vk chain mutant) |
| 16 | Q52L (Vk chain mutant) |
| 17 | P69S (Vk chain mutant) |
| 18 | FGGGTEVVVK |
| 19 | WGPGTLVTVSS |

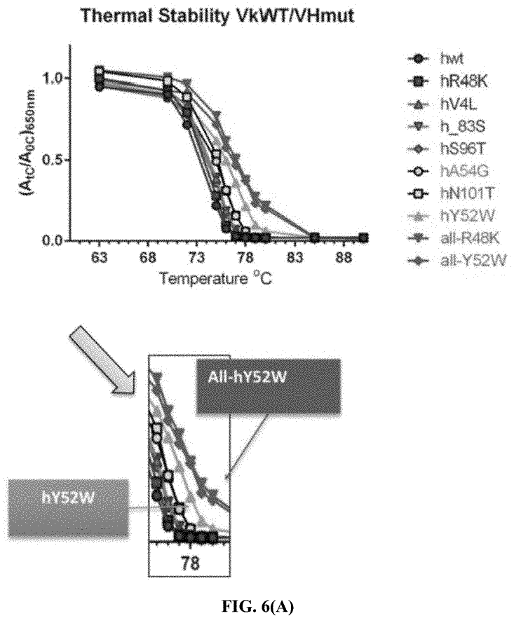

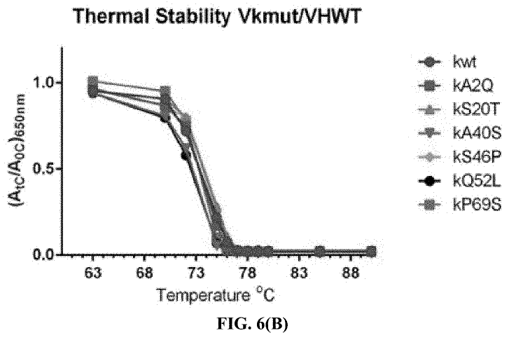

Notably, the clone with all the universal or normalized VH template's changes (FIG. 6, “h.all”) has the same affinity as the wild-type antibody.

In contrast, as shown in FIG. 6C, while the Vk single mutations did not affect binding, the combination of all Vk mutations (“k.all”) abolished binding. It is hypothesized that at least two of the Vk mutations were synthetic lethal and through testing have been identified as A40S and Q52L. As shown in FIG. 6D, k.all without Q52L (-Q52L) and k.all without A40S (-A40S) did not restore the binding of Vk with all mutations (k.all), whereas k.all without both A40S and Q52L (-A40S-Q52L) restored the binding to the similar levels as that of the wild-type Vk (kwt).

A 3 μg/ml solution of goat-anti-rabbit IgG (obtained from Jackson ImmunoResearch Laboratories, Inc. of West Grove, Pa. (“Jackson Imm”); 411-005-003) in phosphate buffer saline (“PBS”) was added to the wells of a round bottom microtiter plate (Greiner 650061) in an amount of 100 μl/well. The plate was then sealed and stored overnight at 4° C.

Wells were emptied and refilled with 150 μl 1.13% bovine serum albumin (“BSA”) in PBS and incubated for an hour at room temperature with shaking. Wells were then emptied and 100 μl/well of serial dilutions of standard Chromopure Rabbit IgG whole molecule (obtained from Jackson Imm; 011.000.003) and sample culture supernatants were added.

Standard and unknowns were allowed to bind for two hours at room temperature with shaking. Wells were then emptied and washed at least four times with PBS+0.5% Tween 20 (“PBST”). Horseradish peroxidase (“HRP”)-conjugated goat-anti-Rabbit IG (Jackson Imm 111-035-046) was diluted 1/2000 in PBS and added in an amount of 100 μl/well.

The HRP-conjugate was incubated for 45 minutes at room temperature with shaking. Wells were then emptied and washed at least four times with PB ST.

TMB (Thermo Fisher cat #002023)) was added in an amount of 100 μl/well and the plate was incubated at room temperature without shaking until the development of blue color stabilized (after approximately 30 minutes).

Signal was read at 650 nm using a microtiter plate reader set to shake the plate for 3-5 seconds before reading.

Antibody binding curves were generated following the ELISA protocol described above except that microtiter plates were initially prepared by plating 100 μl/well of a solution of 0.5 μg/ml of specific antigen in PBS.

### Example 3—Thermal Stability Analysis

To test stability of the above-described mutant and wild-type sequences, thermal stability was selected as a desirable property for optimization.

Thermal stability can be quickly and accurately measured. Lack of thermal stability can lead to additional problems associated with antibodies, such as aggregation or degradation.

Cell culture supernatants were diluted to 10 ng/ml and 20μ aliquots were dispensed into 8-tube PCR strips. Each strip contained up to eight different cell culture supernatants. Several strips were prepared identically, spun on PCR strip centrifuge, and placed in ice.

A thermocycler was programed with several successive 10-minute temperature holds. Strips were placed in the thermocycler in succession, each time removing the previous strip and replacing it with a new one, which, following the incubation, was centrifuged and placed back in ice.

The control strip was left in ice and treated strips were stored at 4° C. O/N.

ELISAs were performed on the following day using a microtiter plate coated with specific antigen.

It was observed that several single mutations in the VH improved thermal stability. Notably, as shown in FIG. 6A, the point mutation Y52W especially resulted in improved thermal stability. However, the stability benefits are additive; maximum stability was achieved even in the absence of Y52W, provided that all other mutations were simultaneously included.

FIG. 6B shows thermal stability curve for WT VH and Vk mutants. The results show Vk variants conferred little or no improvement. FIG. 6C shows xCD2 WT versus the most mutated VH and Vk sequences. The results show maximum stability was achieved by combining the completely mutated VH and the Vk with the most allowed mutations.

The improved stability was unexpected because modification of residues far from binding site can have a significant effect on binding affinity.

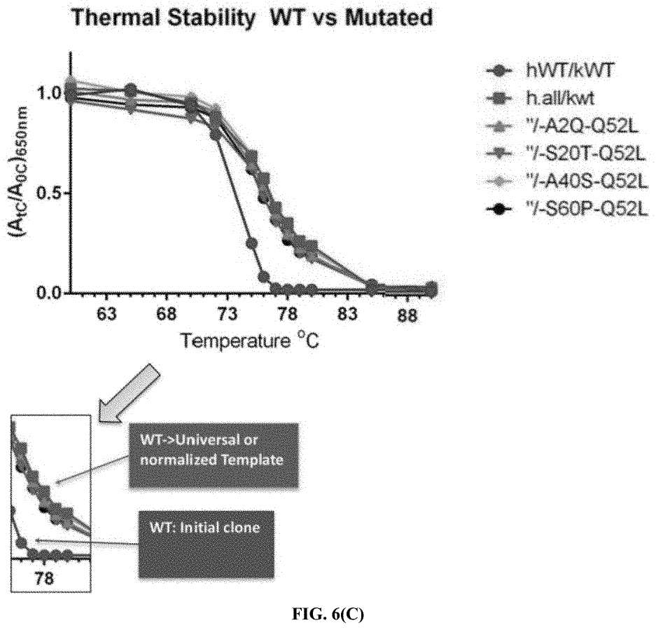

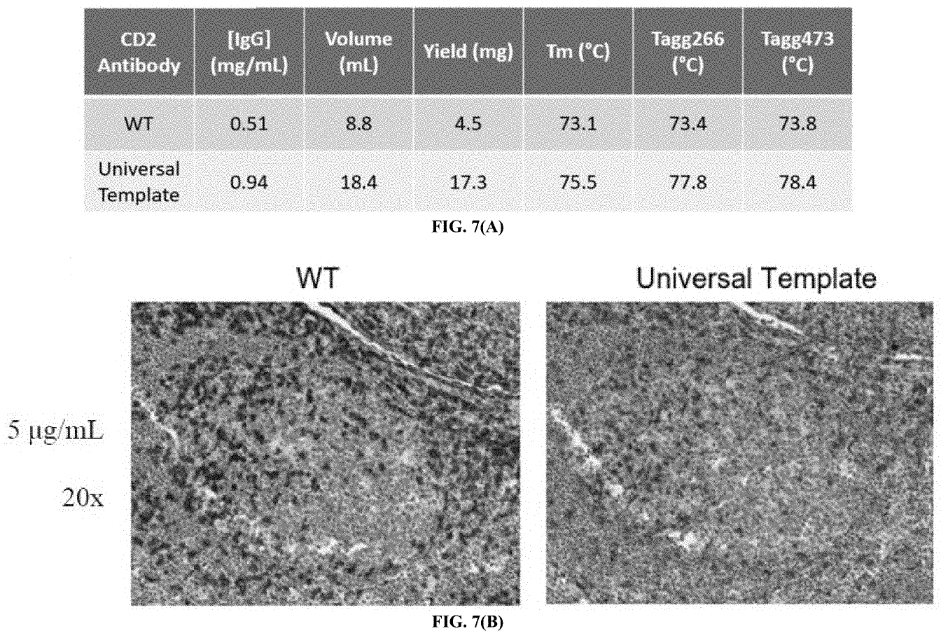

Conversion to the universal or normalized template also results in increased expression levels and Tₘ. FIG. 7A shows that preparing antibodies resulted in more yield of universal or normalized template antibody (17.3 mg) than that of the wild type antibody (4.5 mg), i.e., 3× higher yield with universal or normalized template clone; higher Tₘ of universal or normalized template antibody (75.5° C.) than that of the wild type antibody (73.1° C.), higher T_{agg}266 of universal or normalized template antibody (77.8° C.) than that of the wild type antibody (73.4° C.), and higher T_{agg}473 of universal or normalized template antibody (78.4° C.) than that of the wild type antibody (73.8° C.). FIG. 7B shows the staining intensity of universal or normalized template antibody is comparable to that of wild type antibody.

Advantages of the present invention may include recombinant antibodies that provide a reliable source of product and a starting point for protein engineering efforts to improve bioconjugation, stability, and manufacturability.

### REFERENCES

The below references are incorporated by reference herein in their entirety.
LeFranc M P, et al., IMGT unique numbering for immunoglobulin and T cell receptor variable domains and Ig superfamily V-like domains. Dev. Comp. Immunol. 2003; 2(1): 55-77.
Honegger and Pluckthun, Yet another numbering scheme for immunoglobulin variable domains: an automatic modeling and analysis tool. J. Mol. Biol. 2001; 309(3): 657-70.
Chothia et al., 1998, “Structural determinants in the sequences of immunoglobulin variable domain,” J Mol Biol. 278(2): 457-479.
Narciso J E et al., Analysis of the antibody structure based on high-resolution crystallographic studies. N. Biotechnol. 2011; 28(5): 435-47.
Schuck, P. Reliable determination of binding affinity and kinetics using surface plasmon resonance biosensors. Current Opinion in Biotechnology 8, 498-502 (1997).
Gauglitz, G. Direct optical detection in bioanalysis: an update. Analytical and Bioanalytical Chemistry 398, 2363-2372, doi: 10.1007/s00216-010-3904-4 (2010).
Friguet, B., Chaffotte, A. F., Djavadi-Ohaniance, L. & Goldberg, M. E. Measurements of the true affinity constant in solution of antigen-antibody complexes by enzyme-linked immunosorbent assay. Journal of Immunological Methods. 1985; 77, 305-319.
Vermeer and Norde, The thermal stability of immunoglobulin: unfolding and aggregation of a multi-domain protein. Biophys. J. 2000; 78(1): 394-404.
Svilenov et al., Isothermal chemical denaturation as a complementary tool to overcome limitations of thermal differential scanning fluorimetry in predicting physical stability of protein formulations; Biopharmaceutics. 2018; 125: 106-113.
Strutz et al. Exploring protein stability by NanoDSF; Biopysical J. 2016; 110(3): 393a).
Amin et al., Protein aggregation, particle formation characterization & rheology. Curr. Op. in Colloid & Interface Science. 2014; 19(5): 438-449).
Devereux et al., Nucl. Acids Res. 1984; 12:387.
Neddleman and Wunsch, J. Mol. Biol. 1970; 48:443.
Smith and Waterman, Adv. Appl. Math, 1981; 2.482.
Yonglei Shang, Devin Tesar, Isidro Hötzel. Modular protein expression by RNA trans-splicing enables flexible expression of antibody formats in mammalian cells from a dual-host phage display vector. Protein Engineering, Design and Selection, Volume 28, Issue 10, Oct. 1, 2015, Pages 437-444.
Tesar D, Hötzel I. A dual host vector for Fab phage display and expression of native IgG in mammalian cells. Protein Eng Des Sel. 2013 October; 26(10):655-62. doi: 10.1093/protein/gzt050. Epub Sep. 24, 2013.
Yan, Analysis of oxidative Modification of Proteins. Curr. Protoc. Protein Sci. 2009; 56:14.4.1-14.4.28

1. A method of making a universal or normalized rabbit antibody, the method comprising:
(a) selecting a wild type rabbit antibody;
(b) comparing the sequence of the wild type antibody to the sequence of a normalized antibody, wherein the sequence of the normalized antibody comprises a set of amino acid residues in the variable framework and/or joining regions that are most frequently-occurring at the same position in rabbit antibodies known to be thermally stable and/or having a long-term stability of at least 12 months, and identifying one or more amino acid residues of the wild type antibody that differ from the sequence of the normalized antibody; and
(c) mutating the one or more amino acid residues of the wild type antibody identified in step (b) to the corresponding amino acid residue of the sequence of the normalized antibody to obtain a universal or normalized rabbit antibody, wherein the universal or normalized rabbit antibody comprises one or more mutations generated by the mutating of the one or more amino acid residues of the wild type antibody,
wherein the binding affinity of the universal or normalized rabbit antibody to its target remains within an acceptable range when compared to the binding affinity of the unmodified wild type antibody.

2. The method of claim 1, wherein the universal or normalized rabbit antibody comprises an amino acid sequence of any one of the amino acid sequences of a group consisting of SEQ ID NOs: 1, 4, 5, 6, 7, 8, 9, and 10 in the VH chain.

3. The method of claim 1, wherein the universal or normalized rabbit antibody comprises an amino acid sequence of any one of the amino acid sequences of a group consisting of SEQ ID NOs:2, 12, 13, 14, 15, 16, and 17 in the Vk chain.

4. The method of claim 1, wherein the universal or normalized rabbit antibody comprises:
(a) an amino acid sequence of any one of the amino acid sequences of a group consisting of SEQ ID NOs:1, 4, 5, 6, 7, 8, 9, and 10; and
(b) an amino acid sequence of any one of the amino acid sequences of a group consisting of SEQ ID NOs:2, 12, 13, 14, 15, 16, and 17.

5. The method of claim 1, wherein the universal or normalized rabbit antibody exhibits improved thermal stability as compared to the wild type rabbit antibody.

6. The method of claim 1, wherein the universal or normalized rabbit antibody exhibits improved long-term stability as compared to the wild type rabbit antibody.

7. The method of claim 1, wherein the universal or normalized rabbit antibody exhibits improved deamination as compared to the wild type rabbit antibody.

8. The method of claim 1, wherein the universal or normalized rabbit antibody exhibits improved expression as compared to the wild type rabbit antibody.

9. The method of claim 1, wherein the universal or normalized rabbit antibody is a primary rabbit antibody.

10. The method of claim 1, wherein the one or more mutations is in the J1 region of the kappa chain.

11. The method of claim 1, wherein the one or more mutations is in the FR4 region of the kappa chain.

12. The method of claim 1, wherein the one or more mutations is in the J2, J4, and/or J regions of the heavy chain.

13. The method of claim 1, wherein the binding affinity of the universal or normalized rabbit antibody to its target remains within an acceptable range when compared to the binding affinity of the unmodified wild type antibody.

14. The method of claim 1, wherein the one or more mutations is in a complementarity determining region.

15. A universal or normalized rabbit antibody obtained by the method of any one of claims 1-14.

16. A universal or normalized rabbit antibody comprising an amino acid sequence selected from the group consisting of SEQ ID NOs:1, 4, 5, 6, 7, 8, 9, and 10, in the VH region.

17. A universal or normalized rabbit antibody comprising an amino acid sequence selected from the group consisting of SEQ ID NOs:2, 12, 13, 14, 15, 16, and 17, in the Vk region.

18. A universal or normalized rabbit antibody comprising an amino acid sequence in the VH region that is selected from the group consisting of SEQ ID NOs:1, 4, 5, 6, 7, 8, 9, and 10, and an amino acid sequence in the Vk region that is selected from the group consisting of SEQ ID NOs:2, 12, 13, 14, 15, 16, and 17.

19. The method of claim 1, wherein the universal or normalized rabbit antibody has one or more improved properties appropriate for diagnostic use.

20. The method of claim 19, wherein the one or more improved properties are selected from the group consisting of: thermal stability; long-term stability; decreased aggregations; and decreased oxidation/deamidation.

21. The method of claim 1, wherein the one or more mutations are not in the complementarity region.

22. A method of improving one or more properties in an antibody for diagnostic use, the method comprising:
(a) selecting a wild type antibody that is deficient in one or more characteristics selected from the group consisting of: recombinant expression, thermal stability, long-term stability, aggregation, and oxidation/deamidation;
(b) comparing the sequence of the wild type antibody to the sequence of a normalized antibody, wherein the sequence of the normalized antibody comprises amino acid residues in the variable framework and/or the joining regions that are most frequently-occurring amino acid residues at the same position in a set of rabbit antibodies known to have the one or more characteristics recited in step (a) within an acceptable range, and identifying one or more amino acid residues in the sequence of the wild type antibody that differ from the sequence of the normalized antibody; and
(c) mutating the one or more amino acid residues of the wild type antibody identified in step (b) to the corresponding amino acid residue of the sequence of the normalized antibody, wherein the one or more characteristics recited in step (a) are improved relative to the unmodified wild type antibody.

23. A method of manufacturing a recombinant rabbit monoclonal antibody, the method comprising:
(a) selecting a wild type rabbit antibody;
(b) comparing the sequence of the wild type rabbit antibody to the sequence of a normalized antibody, wherein the sequence of the normalized antibody comprises amino acid residues in the variable framework and/or joining regions that are most frequently-occurring at the same position in a set of rabbit antibodies known to have a characteristic within an acceptable range, wherein the characteristic is selected from a group consisting of: thermal stability; long-term stability; decreased aggregation; decreased oxidation/deamidation, and identifying one or more amino acid residues in the wild type rabbit antibody that differ from the sequence of the normalized antibody;
(c) preparing a standard expression vector encoding an antibody that comprises a complementarity determining region, wherein the complementarity determining region contains alterations to the wild type sequence in the one or more amino acid residues identified in step (b);
(d) expressing the vector of step (c) in a suitable host cell; and
(e) purifying the antibody from step (d).

24. A method of any one of claims 1-14 and 19-23, wherein the set of amino acid residues of the normalized sequence are the most frequently-occurring at the same position in at least 50, at least 100, at least 150, at least 200, at least 250, or at least 300 rabbit antibodies having the relevant characteristic.

25. (canceled)

26. (canceled)

27. (canceled)

28. (canceled)

29. (canceled)
